(19) United States
(12) Patent Application Publication
(10) Pub. No.: US 2025/0141261 A1
(43) Pub. Date: May 1, 2025

(54) SYSTEMS AND METHODS FOR SURGICAL SYSTEM BATTERY MANAGEMENT

(71) Applicant: Alcon Inc., Fribourg (CH)

(72) Inventors: Dan DURANT, Rancho Santa Margarita, CA (US); Rafik KHALILI, Yorba Linda, CA (US); Vivian VUONG, Hacienda Heights, CA (US); Anbumani THANGARAJ, Lake Forest, CA (US); Satish Kumar Calasapakkam GOPAL, Jurupa Valley, CA (US)

(21) Appl. No.: 18/914,702

(22) Filed: Oct. 14, 2024

Related U.S. Application Data
(60) Provisional application No. 63/594,918, filed on Oct. 31, 2023.

(51) Int. Cl. H02J 9/06; A61B 17/00; H01M 10/42; H01M 10/44; H02J 7/00

(52) U.S. Cl. CPC H02J 9/068; A61B 17/00; H01M 10/425; H01M 10/441; H02J 7/0016; H02J 7/0034; H02J 7/00712; A61B 2017/00734; H01M 2010/4271; H01M 2220/30

(57) ABSTRACT

A surgical system including battery packs, a power subsystem and a controller is provided. The power subsystem is configured to connect at least two battery packs in parallel during a backup mode, disconnect the battery packs in a non-backup mode, and prevent each battery pack from charging one or more other battery packs during parallel operation. The controller is configured to individually charge each battery pack to a power capacity equal to or less than a predetermined power capacity, such as 100 Wh.

### CROSS-REFERENCE TO RELATED APPLICATIONS

This application claims the benefit of U.S. Provisional Application Ser. No. 63/594,918 (filed on Oct. 31, 2023), the content of which is incorporated herein by reference in its entirety.

### BACKGROUND

Cataract surgery involves removing a cataractous lens and replacing the lens with an artificial intraocular lens (IOL). The cataractous lens is typically removed by fragmenting the lens and aspirating the lens fragments out of the eye. The lens may be fragmented, for example, using a phacoemulsification probe, a laser probe, or another suitable instrument. During the procedure, the probe fragments the lens, and the fragments are aspirated out of the eye through, for example, a hollow needle or cannula. Throughout the procedure, irrigating fluid is pumped into the eye to maintain an intraocular pressure (IOP) and prevent collapse of the eye.

During cataract surgery, however, AC (Alternating Current) power failure may occur. Such AC power failure may cause harm to a patient undergoing cataract surgery. However, conventional surgical consoles have a number of significant shortcomings including, for example, an inability to maintain a secure power delivery connection with the surgical console, among others.

### SUMMARY

Embodiments of the present disclosure provide a surgical system including a plurality of battery packs, a power subsystem, and a controller. The power subsystem is configured to connect at least two battery packs in parallel during a backup mode, disconnect the battery packs in a non-backup mode, and prevent each battery pack from charging one or more other battery packs during parallel operation. The controller is configured to individually charge each battery pack to a power capacity equal to or less than a predetermined power capacity, such as 100 Wh (watt-hours).

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is noted, however, that the appended drawings illustrate only some aspects of this disclosure and the disclosure may admit to other equally effective embodiments.

FIG. 1A illustrates an exemplary ophthalmic surgical system, in accordance with certain embodiments.

FIG. 1B depicts a system block diagram of the exemplary ophthalmic surgical system of FIGS. 1A and 1B, in accordance with certain embodiments.

FIG. 2 depicts another system block diagram of the exemplary ophthalmic surgical system of FIGS. 1A and 1B, in accordance with certain embodiments.

FIG. 3 depict a flow diagram for battery pack charging, in accordance with certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

The present disclosure will now be described in detail with reference to the drawings, which are provided as illustrative examples of the disclosure so as to enable those skilled in the art to practice the disclosure. Notably, the figures and examples below are not meant to limit the scope of the present disclosure to a single embodiment, but other embodiments are possible by way of interchange of some or all of the described or illustrated elements. Moreover, where certain elements of the present disclosure can be partially or fully implemented using known components, only those portions of such known components that are necessary for an understanding of the present disclosure will be described, and detailed descriptions of other portions of such known components will be omitted so as not to obscure the disclosure.

The present disclosure relates generally to ophthalmic surgical consoles, components therefor, and methods of use thereof during certain ophthalmic surgical procedures. The evolution of medical and surgical technologies has brought about a substantial increase in power demands to facilitate more efficient procedures. In the domain of surgical console systems, there is a lack of power systems for a power budget delivery that may be advantageous for surgical console systems where the power budget system can deliver, for example, a peak power of 625 W (watts) for a duration of 2 seconds and provide a backup time of 5.5 minutes (other power budget system capacities may also be used).

To cater to the power delivery demand of surgical console systems, a capacity of 175 Wh within a rechargeable battery pack (“battery pack” hereinafter) may be used (other battery pack capacities may also be used). However, the practicality of using a single 175 Wh battery pack becomes a challenge, for example, due to the limitations set by current Air Freight regulations, which restrict the power capacity of a single hand-carried battery pack to no more than 100 Wh. This constraint proves significant during field installations where engineers need to hand-carry these components. To circumvent such issues, the embodiments herein utilize a power delivery system having two battery packs, each with a power capacity less than or equal to a predetermined power capacity, such as 100 Wh, etc. Such dual battery pack architecture not only adheres to the current Air Freight regulations but also satisfies the system's power demand by paralleling the two packs for an efficient utilization of the current from both units. In addition, the embodiments herein may be used with battery packs of other sizes.

Importantly, while current Air Freight regulations may restrict the power capacity of a single hand-carried battery pack to no more than 100 Wh, these regulations may be eased in the future, allowing for the power capacity of a single hand-carried battery pack to exceed 100 Wh. Accordingly, the predetermined power capacity may be increased to match the new Air Freight regulation for power capacity, such as 120 Wh, 150 Wh, 200 Wh, etc.

In certain embodiments, beyond merely integrating two battery packs, a power management controller may be implemented for managing and equalizing the power load across both packs. For example, a power controller may implement a method that includes charging one lithium-ion battery pack at a time with a constant current before switching over to charge a second battery pack. In doing so, the power controller ensures that the State of Charge (SoC) between the battery packs remains within, for example, a 5% variance before initiating the charge for the second battery pack. Such power charging protocol ensures both battery packs are equally charged, within, for example, a 5% SoC range, and balanced to meet the system's power demands. Other SoC ranges may also be used (e.g., 1%, 3%, 7%, 10%, 15%, etc.).

In certain embodiments, to promote longevity of the battery packs, the power management controller is configured to charge each pack only until it reaches a predetermined SoC limit (for example, 80%) (other upper limits may also be used). While 80% is used frequently throughout as an example predetermined SoC limit, the predetermined SoC limit may be, for example, 50%, 60%, 70%, 75%, 85%, 90%, 95%, etc. In some embodiments, the predetermined State of Charge may be predetermined by a user, predetermined during surgical console manufacturing, etc. The power management controller monitors and manages the upper limit of SoC during the charging protocol by either charging or discharging the battery packs, thereby effectively maintaining the SoC within, for example, a set 5% range. Advantageously, the power management controller is not restricted to managing only two battery packs. The embodiments described herein may be extended to manage any number of lithium-ion battery packs. Such scalability renders the embodiments herein highly suitable for meeting the high-power backup demand of future surgical products that employ the power delivery framework described herein for improved power solutions in ophthalmic surgical console systems.

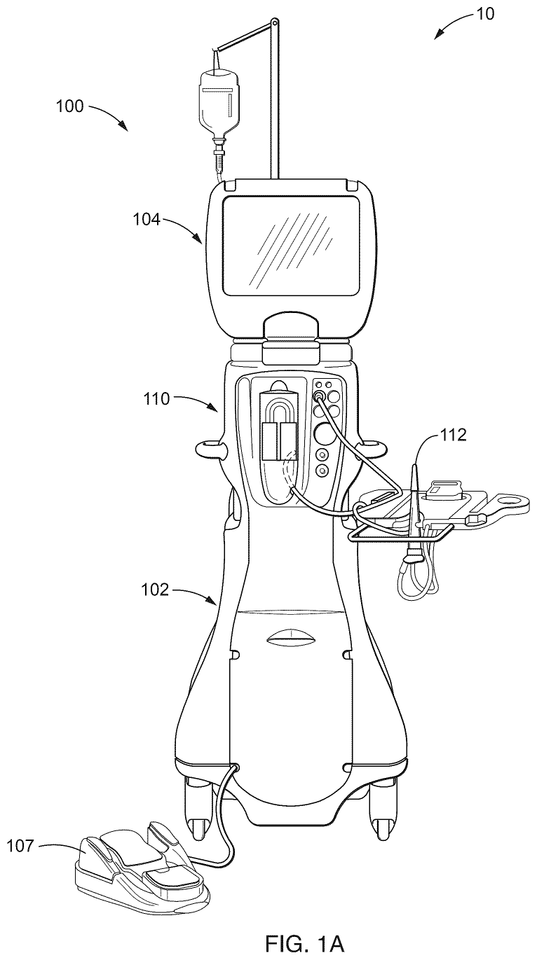

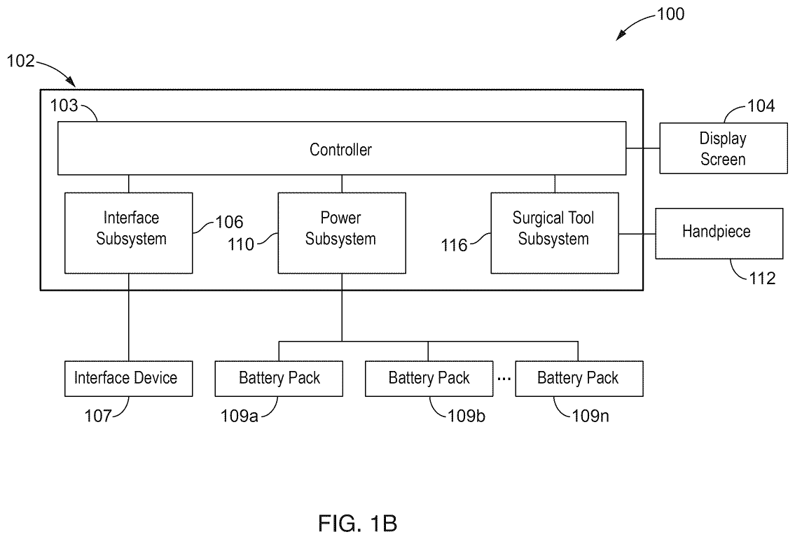

Referring now to FIGS. 1A and 1B, FIG. 1A illustrates ophthalmic surgical console system 10 that may be used to perform ophthalmic procedures on an eye, according to certain embodiments. In the illustrated embodiments ophthalmic surgical console system 10 is interchangeably referred to as “console 100”. FIG. 1B depicts a system block diagram corresponding to console 100. As shown, console 100 includes interface device 107 (e.g., a foot pedal), and handpiece 112. Console 100 includes housing 102, which accommodates power management controller 103 in communication with display screen 104, interface subsystem 106, power subsystem 110, surgical tool subsystem 116, and display screen 104.

For ophthalmic surgical console systems, one of the significant challenges is meeting the system's power budget, which may, for example, use 625 W peak power for a duration of 2 seconds and a backup time of 5.5 minutes. Such power budget requirements may be met using a battery pack of 175 Wh capacity (or larger). However, a configuration utilizing a single 175 Wh battery pack poses a logistical issue due to current Air Freight regulations. Such regulations cap the power capacity of a hand-carried battery pack at 100 Wh, presenting a problem during field installations where these components must be transported by engineers.

Accordingly, the embodiments herein provide a power delivery framework for surgical console systems that employ two or more battery packs, each with a power capacity less than or equal to, for example, 100 Wh. Such approach adheres to current Air Freight regulations while meeting the power demand of the system by connecting the battery packs in parallel. As noted above, when the current Air Freight regulations are eased, the power capacity of each battery pack may be commensurately increased.

Advantageously, the embodiments described herein effectively use the current from multiple battery packs and balance the load on such battery packs with a reliable and efficient power management controller 103 (or simply “controller 103” hereinafter). Controller 103 of the embodiments herein advantageously ensures equal charging between the battery packs while limiting the SoC to prolong the battery life. Controller 103 facilitates efficient charging of individual battery packs while maintaining load balance. Controller 103 is configured to charge one battery pack 109 at a time, ensuring that the SoC between battery pack 109a is within, for example, 5% (or, for example, 1%, 2%, 3%, 8%, 10%, etc.) before initiating the charge for the second battery pack 109b. Controller 103 may also cap the charging process at, for example, 80% SoC (or, for example, 70%, 75%, 85%, 90%, 95%, etc.) to prolong the battery life, which is described in further detail below. Furthermore, controller 103 is scalable and can be extended to any number of battery packs 109, such as battery packs 109a, 109b, . . . , 109n.

In the embodiments described herein, battery packs 109 may advantageously play a significant role in ensuring compliance with transport regulations capacity constraints due to current Air Freight Regulations. As is known, the aviation industry has established guidelines concerning the transport of lithium batteries. One primary concern is the power capacity of a battery pack. The current Air Freight regulations mandate that any hand-carried battery pack should not exceed 100 Wh in capacity. This limitation is rooted in safety concerns, as larger batteries may pose more significant hazards in the event of malfunctions or damages.

Given these constraints, battery packs 109 may be designed with a power capacity of less than or equal to 100 Wh each. Such power capacity ensures compliance with current Air Freight regulations, making battery packs 109 safe and convenient for transport by field engineers or any personnel needing to relocate the equipment. While the individual capacity of each battery pack may be restricted to 100 Wh, when connected in parallel, battery pack 109 may provide the combined power needed to meet surgical console system requirements without breaching transport regulations. In certain embodiments, to further assist with the ease of transport and ensure safety, each battery pack 109 may be ergonomically designed and built to be compact and lightweight, making battery packs 109 easy to handle and carry without causing fatigue. Battery packs 109 each include a high energy density such that despite a smaller size and lower weight, battery packs 109 may store a significant amount of energy, which is advantageous for devices like surgical console systems that require a reliable power source, such as system 10.

Power delivery from each battery pack 109 is optimized to provide consistent energy. When connected in parallel, system 10 may draw current from multiple packs uniformly, ensuring efficient and balanced power distribution to meet peak power requirements. In certain embodiments, each battery pack is equipped with an advanced charge controller. This controller ensures the battery is charged to its optimum capacity (for example, up to 80% SoC) to enhance longevity and maintain health. Other optimum capacities are also contemplated (e.g., up to 70%, 75%, 85%, 90%, 95%, etc.).

In alignment with aviation safety standards, the battery packs may be embedded with safety features like overcharge protection, over-discharge protection, short circuit protection, and thermal protection. These features prevent any potential hazards that can arise from battery malfunctions. While other battery types may be implemented, lithium-ion battery packs are especially well suited for medical applications as the battery chemistry provides multiple advantages. For example, lithium-ion batteries are known for their high energy density, low self-discharge, and long cycle life, making them ideal for demanding applications.

Each battery pack 109 may be designed to have extended operational life facilitated by power management protocol of controller 103. For example, by limiting the charge to a predetermined SoC (e.g., 80% SoC), the wear and tear on the battery cells of battery packs 109 are significantly reduced, ensuring a more extended period before the battery pack needs replacement. One of the advantageous features of battery packs 109 is a modular design which allows for easy replacement or addition of packs as necessary, making system 10 adaptable and scalable. Depending on specific power needs, more battery packs may be integrated seamlessly. Moreover, understanding the need for uninterrupted power, especially in critical applications, battery packs 109 may be designed for quick swapping. Such swapping feature allows a depleted battery to be replaced rapidly without shutting down the system.

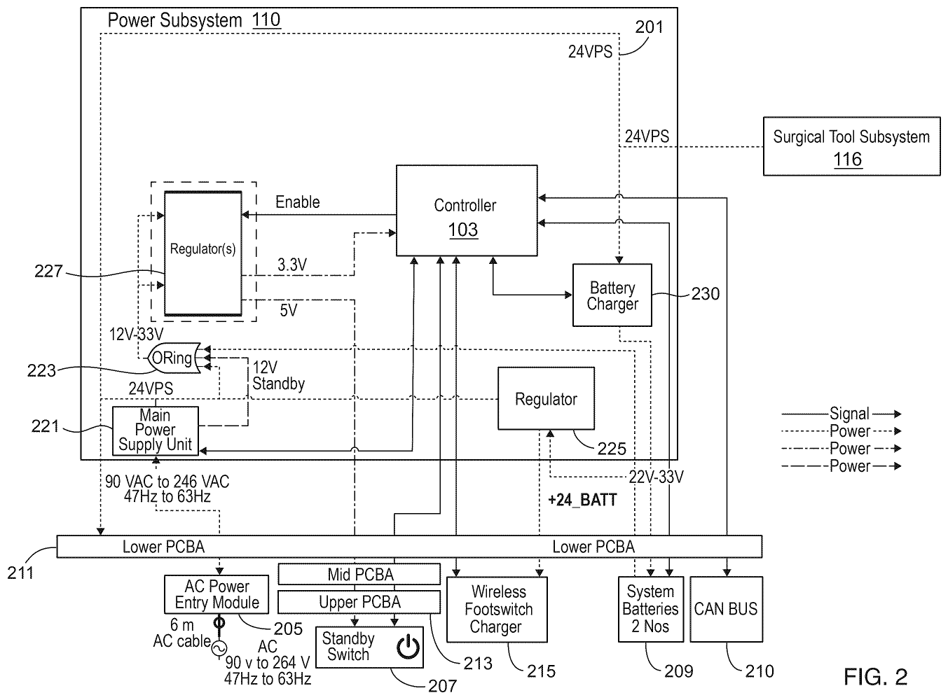

Referring now to FIG. 2, in conjunction with FIGS. 1A and 1B, FIG. 2 depicts another system block diagram of ophthalmic surgical system 10, in accordance with one or more embodiments. As shown, controller 103 is in communication with certain components of power subsystem 110, and together control and operate system 10. In certain embodiments, power subsystem 110 provides console 100 components, such as surgical tool subsystem 116, etc., with clean, stable, sufficient power to reliably operate surgical consoles under varying AC means and environmental conditions, for example as expected in worldwide hospital environments.

As shown in FIG. 2, surgical console 100 implements a modular architecture, wherein each module power is provided by, for example, a single primary 24 VDC (Direct Current bus voltage) bus 201 distributed throughout system 10. In certain embodiments, power subsystem 110 includes, inter alia, AC power entry module 205, standby switch 207, battery packs 209 (such as battery packs 109a, 109b), and power distribution through several PCBAs (printed circuit board assemblies).

In certain embodiments, power subsystem 110 receives rated input power from AC mains, such as 100 VAC (Volts Alternating Current) to 240 VAC, 50/60 Hz (Hertz), etc. Other input power from AC mains may also used/received (e.g., depending on the power grid configuration/country the power system 110 is located in). Power subsystem 110 may deliver up to, for example, 918 W of continuous power at +24 VDC in all, or nearly all, operational conditions. Other power metrics are also contemplated.

For example, in an event of AC mains failure, battery packs 209 deliver power needed to maintain operation of a surgical console for a selected period of time (e.g., in some embodiments, at least 625 W of continuous power may support normal system functionality at least for 1.5 seconds). (Other battery pack sizes and time periods are also contemplated (e.g., with systems of different sizes/power needs)). In certain embodiments, power subsystem 110 may accept input from standby switch 207 and then drive a backlight of standby switch 207 to indicate the various system power modes of operations, discussed further below. In certain embodiments, power subsystem 110 may monitor the health of the power distribution (such as voltage and current) to ensure safe operation, manages battery power management (such as charging and discharging). Power subsystem 110 delivers power for wireless footswitch chargers 215 and surgical tool subsystem 116, based on the input from an upstream supervisor PCBAs 211, 213. Power subsystem 110 communicates the status of power subsystem to the upstream supervisor PCBAs.

In certain embodiments, power subsystem 110 includes AC power entry module 205. In certain embodiments, AC power entry module 205 receives rated AC mains input (e.g., 100 VAC to 240 VAC/50 Hz to 60 Hz) from a wall socket through the power cord into an inlet socket (such as an IEC (International Electrotechnical commission) 60320 Type C20, etc.) and switches the filtered AC input to console 100. For example, in certain embodiments, such switching and filtering may be facilitated via a J30 connector in a Lower PCBA 211.

In certain embodiments, AC power entry module 205 has an inbuilt medical grade EMI (Electro Magnetic Induction) filter with very low leakage current (e.g., <5 μA) (microAmps) which suppresses the electromagnetic noise transmitted through conduction. In certain embodiments, AC power entry module 205 may include a 2-pole (Line and Neutral) circuit breaker switch that switches the filtered AC into the console.

In certain embodiments, AC power entry module 205 may be advantageously rated to handle 20A of current @250 VAC (other power entry module ratings may also be used). A dielectric test voltage rating of AC power entry module 205 between the Line/Neutral and Protective Earth terminals may advantageously exceed the electrical isolation requirement of system 10, which may be, for example, 1500 VAC.

As shown in FIG. 2, power subsystem 110 includes main power supply unit (PSU) 221 in communication with Lower PCBA 211, which, in certain embodiments may be in communication via a connector. In certain embodiments, power subsystem 110 may receive AC inputs (Line, Neutral and Earth) from a Lower PCBA 211. In certain embodiments, such AC inputs may be advantageously routed through the PSU 221 and terminated at J4 connector from which PSU 221 receives the AC input through the AC bus bar, as shown in FIG. 2.

In certain embodiments, PSU 221 may accept rated AC mains input (e.g., 100 VAC-240 VAC/50 Hz-60 Hz) and convert such input into Isolated DC output voltages (e.g., 24 VDC, 12 VDC_standby), which drives the secondary circuits of console 100. Such isolated DC output voltages comply with two Means of Patient Protection (2MOPP) and Type BF applied part leakage current requirements (e.g., <500 uA) of IEC 60601-1,3'rd Ed medical safety standard.

When the AC input is in acceptable range, PSU 221 by default delivers 12 VDC_Standby output (secondary voltage). The maximum rated current that can be drawn from 12 VDC_Standby output may be 0.5 A (other maximum rated currents are also contemplated). The 12 VDC_Standby may be used to power the power control circuitry in the power subsystem 110, such as 5V/3.3V DC-DC regulators, controller 103 and standby switch 207.

In certain embodiments, isolated DC output from PSU 221 (24 VPS) may be used as the primary voltage to power the console subsystem modules (such as surgical tool subsystem 116). PSU 221 may deliver, for example, the 24 VDC output with a tolerance, for example, of ±5%. The maximum rated current that can be drawn from the 24 VDC output may be 50 A. Such current equates to 1200 Watts of power that can be delivered from PSU 221. Other PSU sizes, output power, maximum rated currents, and tolerances may also be utilized depending on power needs.

In certain embodiments, PSU 221 advantageously delivers, for example, up to 918 W of continuous output power at, for example, 24 VDC output. In this embodiment, for meeting the 918 W output power at worst case AC input of 90 VAC, a conversion efficiency for PSU 221 may be set to at least 85%. (918 W (output power)/85%=1080 W input power at PSU). Such efficiency equates to 12 A of AC input current drawn by the PSU at 90 VAC (12 A×90 VAC=1080 W). Because the 12 A is 80% of 15 A, therefore console 100 may advantageously operate from any 15 A AC input wall socket in the world. Other conversion efficiencies, input power, and input currents may also be used.

In certain embodiments, PSU 221 has an AC input to DC output conversion efficiency of up to, for example, 94% and delivers, for example, at least 1200 W of continuous output power at 24 VDC output. For example, in certain embodiments, after a switch in AC power entry module 205 is pressed, the 24 VDC output of PSU 221 is enabled by default to subsystems (such as surgical tool subsystem 116) through System Power PCBA hardware even when the System Power PCBA is unprogrammed. When the System Power PCBA is programmed, then the standby switch 207 may be required to be pressed for enabling the 24 VDC output of PSU 221 to subsystem hardware (e.g., surgical tool subsystem 116).

In certain embodiments, PSU 221 may include the following I/O (Input/Output) control and status signals (24 VDC Power good status, 24 VDC Remote ON/OFF control) interfaced to the I/O pins of controller 103. The above I/O pins may be used to sense the health of the 24 VDC output and enable/disable 24 VDC output voltage.

At full load condition, PSU 221 holds, for example, 90% of 24 VDC output at least, for example, 13 ms (milliseconds) from the moment the input AC voltage drops below the acceptable range. (Other full load conditions are also contemplated (e.g., 70%, 80%, 95%, etc. of 24 VDC.) Other time periods are also contemplated (e.g., 5 ms, 10 ms, 15 ms, 20 ms, 50 ms, etc.). In some embodiments, PSU 221 lowers the 24 VDC Power good status I/O signal after 8 ms from the moment the input AC voltage drops below the acceptable range. Other timing (e.g., 5 ms, 7 ms, 10 ms, etc.) is also contemplated. The remaining 5 ms (i.e., 13 ms-8 ms) duration may be used by controller 103 and/or power subsystem 110 to seamlessly switch to momentary battery backup power to support back up power requirements, such as normal system functionality, etc., for example, for 1.5±0.5 seconds.

PSU 221 may support industry standard PMBus (Power Management Bus) command protocol which enables the controller 103 to communicate with the PSU 221 through I2C bus to read one or more parameters of system 10. Certain embodiments may include the following system 10 parameters: AC Input Voltage, DC output voltage (24 VDC, 12 VDC_Standby), AC Input current, DC output current (24 VDC), health of power supply, fan and power supply temperature, overvoltage, and/or overcurrent and voltage out of specification status.

In some embodiments, when the Console receives AC mains voltage from the wall (through turning ON the rocker switch in AC power entry module 205), the PSU may deliver 12 VDC_Standby voltage by default (irrespective of enabling the 24 VDC output from PSU) and feeds into the System Power PCBA through its J8 connector. When the AC is available to PSU from AC power entry module 205, the 24 VDC (24 VPS) from PSU 221 is enabled to the subsystem by default (even without pressing the standby switch) when the System Power PCBA is not programmed. This is achieved by the hardware logic in System Power PCBA which drives the 24 VPS output enable pin low (-PS_INHIBIT) to output 24 VPS from PSU to support powering the host module even when the System Power PCBA is unprogrammed. This feature also helps to program the logic in System Power PCBA from the host module. If the System Power PCBA is programmed, then the Standby switch must be pressed to enable the 24 VDC output of PSU to subsystem hardware (e.g., Host PC (Personal Computer) module).

As shown in FIG. 2, power subsystem 110 includes ORing circuit 223 that combines the DC output from PSU 221 (e.g., 24 VPS, 12 V_Standby) and battery packs 209 (e.g., at 32.4 VDC) to provide system redundancy by paralleling power sources. The output voltage (e.g., 12 VDC to 32.4 VDC) from ORing circuit 223 sources the power regulator 225 to deliver, for example, 3.3 VDC and 5 VDC for circuits (such as controller 103, standby switch 207, and wireless Footswitch 214 proximity sensors). In certain embodiments, one purpose of the ORing circuit 223 is to ensure that the controller 103 has priority on being powered.

As a parallel path, the output voltage (e.g., 12-32.4 VDC) from ORing circuit 223 sources the power regulator(s) 227 to deliver, for example, 3.3 VDC and 5 VDC for circuits such as wireless footswitch charger 215. When console 100 is in Standby or Deep Sleep power mode, in order to consume less power, controller 103, turns OFF the power to the peripherals by disabling power regulators 225, 227.

In certain embodiments, standby switch 207 includes embedded logic. For example, in order to forcefully shutdown the console, when the user presses the console standby switch 207 for more than, for example, 5 seconds (also referred as long press), the controller 103 or a controller of power subsystem 110 may disable the primary DC output of PSU 221 (24 VPS) irrespective of concurrent action. Additionally, standby switch 207 logic may reset controller 103 and prevent the system electronics being powered from battery pack voltage.

In certain embodiments, power subsystem 110 and/or controller 103 may include a 32-bit ARM (Advanced RISC (Reduced Instruction Set Computer) Machine) based microcontroller that monitor, control and communicates with system 10 components. In certain embodiments, controller 103 and/or power subsystem 110 may operate at 40 MHz input clock supplied by an external crystal oscillator. Voltage monitoring circuits monitor the health of the following DC voltages and initiates an interrupt to controller 103 when the voltage drops below, for example, 5% of the respective DC voltages. The scale down version of the below voltages are fed to the ADC (Analog to Digital Converter) pins of the controller 103 for sensing the health of the voltages. In certain embodiments, power subsystem 110 monitors the DC output current (@24 VDC) delivered from PSU 221 and may also monitor the voltage and current from battery pack 209 output and footswitch charger 215.

In certain embodiments, controller 103 communicates with various components through CAN (Controller Area Network) bus 210. In certain embodiments, controller 103 may be programmed via a JTAG (Joint Test Action Group) port (J6). CAN bus 210 may be used to update an ARM bootloader or subsystem application of controller 103. In certain embodiments, controller 103/power subsystem 110 monitors the health of the system voltages through corresponding ADC interfaces, as shown in FIG. 2.

As shown in FIG. 2, the 24 VDC from PSU 221 is fed to the battery charger 230 to boost 24 VDC to 32.4 VDC (nominal) to 36 VDC for charging two or more battery packs 209. Buck regulator 224 scales down the 33 VDC from battery packs to 24 VDC to power system 10 from the battery packs 209 in an event of AC input failure. The outputs of the Boost and buck voltage regulators are enabled/disabled by the GPIO (General Purpose Input/Output) pins of controller 103.

In certain embodiments, controller 103 functions to control the operations of Lower and Upper PCBAs 211, 213. In certain embodiments, Lower and Upper PCBAs 211, 213 may include a dedicated microcontroller, FPGA (Field Programmable Gate Array), etc. In certain embodiments, controller 103 communicates with a Fuel Gauge IC embedded in battery pack 209 through the I2C bus interface. In certain embodiments, the Fuel gauge IC of the two battery packs may have the same device address, such that controller 103 individually communicates with the battery packs through I2C Multiplexer IC, for example, included in a Lower PCBA 211. In certain embodiments, controller 103, using, for example, a GPIO, individually enables charging of battery packs 209. For example, controller 103 monitors the SoC of each of the battery packs 209, and, in certain embodiments, battery packs 209 are charged one at a time.

In certain embodiments, controller 103 starts charging the battery pack when the corresponding SOC drops below 60%. If SoC is above 60% then controller 103 maintains the SoC between each of the battery packs to be always within, for example, 5% (or another selected value such as within 1%, 3%, 10%, 15%, etc.). The battery pack will not be charged when its SoC is over 60% (in this embodiment). For example, in this embodiment, if battery pack 109a's SOC is below 60% and battery pack 109b's SOC is above 60%, then controller 103 will start charging battery pack 109a that is below 60% and bring SoC within 5% of SOC of battery pack 109b, and controller 103 stops charging the battery packs when a SOC reaches, for example, 80% (other values are also possible, for example, 70%, 75%, 85%, 90%, etc.). The power subsystem 110 discharges either battery pack 109a, 109b to a minimum of, for example, 30% SoC before putting the system 10 into deep sleep, preventing further system discharge of battery packs 109a, 109b. Other SoC levels before entering into deep sleep are also possible (e.g., 5%, 10%, 20%, 25%, 35%, etc.). In certain embodiments, controller 103 permits or restricts charging the battery packs 109 when the internal battery pack temperature is between, for example, 5° C. (Celsius) and 45° C. (other restricted charging temperature ranges are also possible depending on, for example, the battery type, environment, etc.).

In power subsystem 110, at a given instance, the maximum DC power that is consumed for charging each battery pack 109 may be limited to, for example, 60 W. Other maximum DC power limits are also contemplated (e.g., 10 W, 20 W, 50 W, 75 W, 100 W, etc.). Battery charger 230 monitors the charging current for battery packs 209. When controller 103 detects that the battery pack draws more than, for example, 60 W for charging, as a safety feature, controller 103 disables the battery charging.

In certain embodiments, battery charger 230 performs trickle charging, in which the battery packs 209 will be charged with a charging current of, for example, 400 mA or less, until the battery voltage becomes, for example, 30 VDC. Subsequent to 30 VDC voltage, the battery packs 209 will be charged with a primary charging current of, for example, 1.5 A. Other trickle charging currents (e.g., 250 mA, 500 mA, 700 mA, etc.) and battery voltage goals (e.g., 20 VDC, 35 VDC, 40 VDC, etc.) are also possible. Other primary charging currents are also possible (e.g., 1 A, 2 A, 2.5 A, 5 A, etc.).

In certain embodiments, controller 103 facilitates switching from direct power supply to battery. In certain embodiments, at a full load condition, the power subsystem 110 can hold, for example, 90% of 24 VDC output at least for 13 ms from the moment the input AC voltage drops below the acceptable range. As noted above, other full load conditions are also contemplated. Power subsystem 110 lowers the 24 VDC power good status I/O signal after 8 ms from the moment the input AC voltage drops below the acceptable range. Such signal triggers an interrupt to controller 103. The remaining 5 ms (i.e., 13 ms-8 ms) duration may be used by controller 103 to seamlessly switch the buck regulated battery packs output voltage (24V_BATT) for powering the console subsystems for 1.5 seconds to ensure normal system functionality.

In certain embodiments, battery charger 230 monitors and regulates the battery charging current and provides an interrupt to the controller 103 when battery charger 230 detects a fault condition during charging of battery packs 209. In certain embodiments, when battery charger 230 detects any fault (such as when the charging power drawn by a battery pack 209 is beyond 60 W), then battery charger 230 enables the red LED (light emitting diode). Battery charger 230 enables the green LED when the battery charging is within acceptable threshold levels.

In certain embodiments, controller 103 and power subsystem 110 ensure that one battery pack 109 does not charge the other when they are connected in parallel by regulating the voltage across each pack. When battery packs 109 are connected in parallel, the voltage is supposed to be the same across each battery.

In some embodiments, both battery packs may be allowed to discharge independently while maintaining the balance between them end ensuring optimal performance. In some embodiments, this involves using one or more diodes. Diodes are semiconductors that allow current to flow in one direction but not the other. In this setup, diodes are strategically positioned to prevent backflow of current from one battery pack to another. Each battery pack 209 in the system is connected to the shared load via one or more diodes that are disposed, for example, in ORing circuit 223. Such placement ensures that the current supplied by each battery pack flows only toward the shared load, and not back toward another battery pack. Consequently, each battery pack operates independently, preventing one battery from charging the other.

Power subsystem 110 also includes real-time monitoring of the voltage across each battery pack. If the voltage difference between the two packs is significant (greater than a certain threshold), the power management controller may intervene and switch the charging process to balance the SoC between the two packs. Switches may advantageously disconnect a battery from the circuit if its voltage drops too low or rises too high. Disconnecting the battery in this manner protects each battery from potentially damaging conditions and aids in maintaining balanced operation.

In certain embodiments power subsystem 110 may be tightly integrated with the power management controller 103. Controller 103 continuously monitors the state of each battery pack and can control the switches based on the feedback it receives, via power subsystem 110. Such functionality ensures an efficient, balanced use of power from both packs, while also protecting the battery packs from damaging conditions and assists in maintaining a balanced operation between the two battery packs. Such balanced operation may extend the life of the battery packs, improve system reliability, and ensure consistent power delivery. By preventing one battery from charging the other and avoiding potentially damaging conditions, power subsystem 110 and controller 103 greatly improves the safety of the system 10.

Controller 103 and power subsystem 110 advantageously operate each battery pack independently, integrate diodes, voltage monitoring and switch controls, maintain the state of charge balance, promote system longevity, and ensure safe operation. Thus, when one battery has a higher charge level (higher voltage), the higher voltage battery may start charging the other, less-charged battery (lower voltage). To avoid this, controller 103 constantly monitors the voltage across each battery pack and adjusts the input or output of each battery to ensure that their voltage levels are equal.

In certain embodiments, controller 103 and power subsystem 110 may manage the switching between immediate battery backup mode and an extended battery backup mode. For example, during an AC power failure, controller 103 first activates the immediate backup mode, where two battery packs are connected in parallel to deliver a peak power output of, for example, 625 W for 2 seconds. After this period, controller 103 checks if AC power is restored. If not, it switches to extended battery backup mode. In this mode, the battery packs deliver a lower power of, for example, 145 W at 24 VDC, thereby extending the backup time to, for example, 5.5 minutes. This switch is triggered based on a preset time limit and on the status of the AC power supply. In certain embodiments, controller 103 is configured to activate extended battery backup mode when the SoC of the battery packs is, for example, 21%.

In certain embodiments, controller 103's decision process for charging one battery pack at a time may be based on the SoC of each battery pack. Controller 103 continually monitors the SoC of each pack and selects the one with the lower SoC for charging first. Charging one pack at a time allows controller 103 to better control the SoC of each pack and maintain a balance between them.

Advantageously, the 5% SoC variation is a buffer set by controller 103 to ensure a balance between the battery packs while still allowing for slight differences in charging and discharging rates. If the SoC difference between the battery packs becomes more than 5%, controller 103 may interpret this as an imbalance and initiate corrective measures, such as charging the lower SoC battery pack or discharging the higher SoC battery pack to maintain balance. However, if the SoC difference is less than 5%, controller 103 interprets this as acceptable variation and continues to monitor the battery packs without taking additional action. Other SoC variations are also contemplated (e.g., 1%, 3%, 7%, 10%, etc.).

The 80% SoC charging limit is set to extend the lifespan of the battery packs. For example, in certain embodiments, battery packs 109 may include lithium-ion batteries, which, while highly efficient, can suffer from stress and potential damage if charged to their maximum capacity repeatedly. By setting the SoC charging limit to 80% SoC, controller 103 helps preserve the health of the batteries, thereby enhancing their lifespan and reliability. Furthermore, this limit also creates a buffer that allows the battery packs to accommodate sudden power needs without overstraining the battery packs. This limit, combined with the balance maintained between the battery packs, ensures efficient power management for the surgical console. Other SoC charging limits may also be used (e.g., 75%, 85%, 95%, etc.).

Controller 103 also predicts and manages power demand. Surgical console 100, given its critical function, cannot afford power interruptions. Therefore, in certain embodiments, controller 103 uses predictive analysis based on historical power consumption data, battery health indicators, and real-time power demand to effectively decide when to switch power sources or initiate charging sequences. Such prediction ensures an uninterrupted power supply to the console while optimizing battery performance.

In addition to battery health and power demand, power subsystem 110 also has safeguards in place to protect the battery packs from potential damage. Controller 103 and power subsystem 110 may continuously monitor battery temperature, voltage, and current to prevent overheating, overcharging, and excessive discharging, which could harm the battery packs. If any parameter goes beyond the safe limits, controller 103 initiates protective measures, which could include reducing the charging rate, disconnecting the battery, or switching to the other battery pack.

Because controller 103 manages multiple battery packs, controller 103 must also manage the potential for uneven wear and tear between battery packs 109. Controller 103 may implement a process to rotate which battery pack charges first or discharges first during usage. Such rotation ensures both battery packs experience similar levels of wear over time, promoting a more balanced lifespan and efficiency. In certain embodiments controller 103 may also play a role in maintenance and fault detection. By monitoring various battery health parameters, controller 103 can detect potential faults or degradation in battery packs 109 including changes in the battery's internal resistance, capacity fade, or unusual temperature variations. Upon detecting such anomalies, controller 103 may alert the user or initiate preemptive measures to prevent potential power failure.

Advantageously, controller 103 may also accommodate scalability, allowing for the inclusion of more battery packs 109 when needed. Such scaling functionality may be particularly valuable for future iterations of the surgical console 100 that might require higher power backup. By maintaining a design that can handle multiple battery packs, controller 103 ensures the console's design remains adaptable and future-proof.

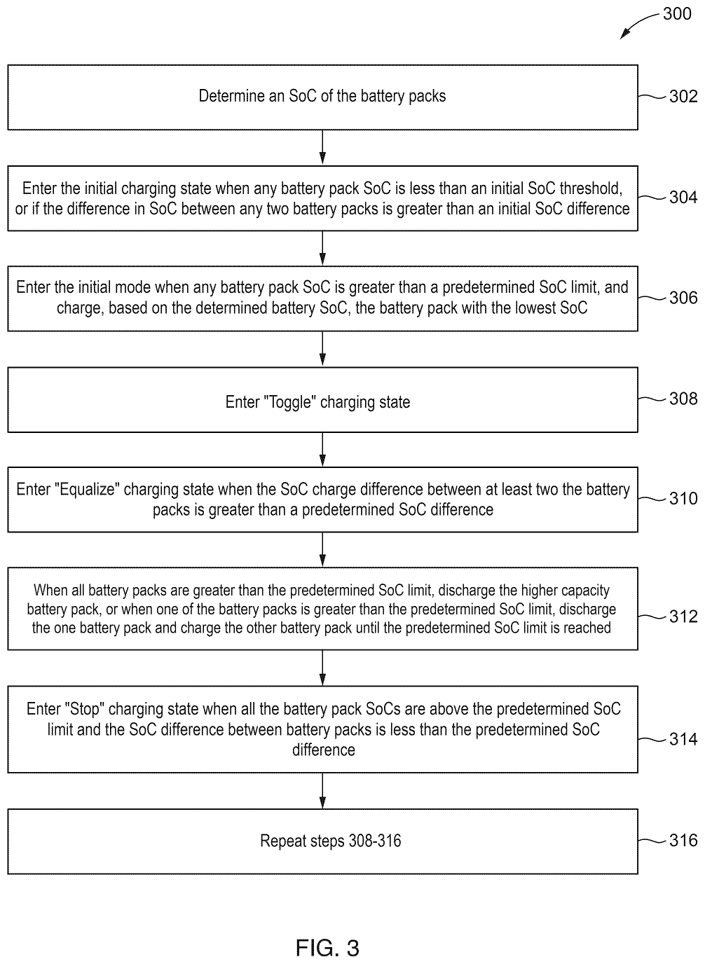

Referring now to FIG. 3, in conjunction with FIGS. 1A, 1B, 2, FIG. 3 depicts a flow diagram corresponding to a charging process 300. Charging process 300 is implemented by system 10 via controller 103 and power subsystem 110. System 10 charging protocol includes four main operational states, INITIATE, TOGGLE, EQUALIZE, and STOP. Such operational states are descried below in the context of blocks 300-314, described in detail below.

At 302, controller 103 determines a condition to start charging by checking if any of the battery packs 109 SoC is less than an initial SoC threshold, such as 60%, or if the difference in the initial SoC between any two battery packs 109 is greater than an initial SoC difference, such as 5%. If either of these conditions is met, controller 103 initiates the charging process and flow proceeds to 304. Other initial SoCs are also contemplated (e.g., 20%, 40%, 70%, 85%, etc.). Other differences in initial SoCs to look for are also contemplated (e.g., 1%, 3%, 7%, 10%, etc.).

At 304, the initial charging state is entered when any battery pack 109 SoC is less than an initial SoC threshold, or if the difference in SoC between any two battery packs 109 is greater than an initial SoC difference.

At 306, controller 103 determines if any battery SoC is greater than a predetermined SoC limit, such as 80%, etc. If any battery's SoC is greater than the predetermined SoC limit, controller 103 starts charging the battery with the lowest SoC and enters the “Toggle” charging state at 308. If not, controller 103 determines if a predetermined SoC difference between at least two battery packs is greater than, for example, 5%. (“80%” and “5%” are given as examples. Other values may also be used.).

In the “Toggle” charging state, controller 103 toggles charging between battery packs 109 when the SoC of the charging battery is greater than the SoC of the idle battery by, for example, 3%. If the predetermined SoC difference between battery packs 109 is greater than, for example, 5%, controller 103 enters the “Equalize” charging state at 310 and flow proceeds to 312. If not, controller 103 remains in the “Toggle” charging state.

At 312, in certain embodiments, in the “Equalize” charging state, controller 103 equalizes the charge between the battery packs 109. If all battery packs 109 are greater than the predetermined SoC limit, such as 80%, etc., controller 103 discharges the higher capacity battery pack 109. If one of the battery packs 109 is greater than the predetermined SoC limit, such as 80%, etc., controller 103 discharges that battery pack and charges the other battery pack 109 until the predetermined SoC limit is reached.

At 314, controller 103 then determines whether all the battery pack SoCs are above the predetermined SoC limit, such as 80%, etc., and whether the SoC difference between battery packs is less than the predetermined SoC difference, such as 5%. If all the battery pack SoCs are above the predetermined SoC limit, such as 80%, etc., and the SoC difference between battery packs is less than the predetermined SoC difference, such as 5%, controller 103 disables battery charging and equalizing, and enters the “Stop” charging state.

At 316, controller 103 returns to the previous step and repeats the process until the stopping condition is met.

In certain embodiments, during charging operations, 310 may follow 302, 312 may follow 310. In some certain embodiments, 302, 310 and 312 may be repeated.

Accordingly, the embodiments described above accommodate scalability, allowing for the inclusion of more battery packs if needed thereby ensuring the console's design remains adaptable and future-proof. The embodiments herein manage and optimize power delivery, battery health, and system reliability, ensuring that surgical consoles perform their critical function without interruption. The capacity for adaptability and scalability of the embodiments herein prepares such surgical consoles for future power demands and potential design upgrades.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

What is claimed is:

1. A surgical system, comprising:
at least two battery packs;
a power subsystem configured to:
connect the at least two battery packs in parallel during a backup mode,
disconnect the at least two battery packs in a non-backup mode, and
prevent each battery pack of the at least two battery packs from charging a different battery pack in the at least two battery packs during parallel operation; and
a controller configured to individually charge each battery pack of the at least two battery packs to a power capacity equal to or less than a predetermined power capacity.

2. The surgical system of claim 1, wherein each battery pack of the at least two battery packs provides an output of 32.4 VDC (Volts Direct Current) and delivers a current of 2.7 Ah (Ampere Hours).

3. The surgical system of claim 1, wherein:
the predetermined power capacity is 100 Wh (Watt hours); and
each battery pack of the at least two battery packs, when fully charged, delivers power up to 87.48 Wh.

4. The surgical system of claim 1, wherein the controller is further configured to limit a state-of-charge (SoC) of each battery pack of the at least two battery packs to 80% during charging.

5. The surgical system of claim 1, wherein the controller is further configured to transition into an immediate battery backup mode during an AC (Alternating Current) input failure, with the at least two battery packs providing a peak pulse discharge power of at least 625 W at 24 VDC for up to 2 seconds.

6. The surgical system of claim 5, wherein the controller is further configured to transition into an extended battery backup mode after the immediate battery backup mode if AC input remains unavailable, supporting limited subsystem powering with capacity to deliver 145 W power at 24 VDC from the at least two battery packs for 5.5 minutes or until the at least two battery packs are depleted.

7. The surgical system of claim 6, wherein the controller is further configured to activate extended battery backup mode when a state-of-charge (SoC) of the battery packs is 21%.

8. The surgical system of claim 1, wherein the controller is further configured to switch operation of the at least two battery packs to parallel mode only when needed to support seamless momentary backup.

9. The surgical system of claim 1, wherein the power subsystem includes one or more diodes to prevent backflow of current between the at least two battery packs.

10. The surgical system of claim 1, wherein the controller is further configured to charge or discharge the at least two battery packs to keep a state-of-charge (SoC) within a set range.

11. A battery management method for a surgical system, comprising:
determining a state-of-charge (SoC) for at least two battery packs;
when a difference between the SoC for the at least two battery packs is greater than a predetermined SoC difference, equalizing the SoCs between the at least two battery packs; and
when the SoC for one battery pack of the at least two battery packs is greater than a predetermined SoC limit and the SoC for a different battery pack of the at least two battery packs is less than the predetermined SoC limit, discharging the one battery pack to the predetermined SoC limit and charging the different battery pack to the predetermined SoC limit,
wherein each battery pack has a power capacity equal to or less than a predetermined power capacity.

12. The battery management method of claim 11, wherein the predetermined power capacity is 100 Wh, the predetermined SoC difference is 5%, and the predetermined SoC limit is 80%.

13. The battery management method of claim 11, wherein determining the SoC includes determining an initial SoC for each battery pack of the at least two battery packs, and the battery management method further comprises:
when the initial SoC for one battery pack of the at least two battery packs is less than 60%, charging the at least two battery packs to 80% SoC; and
when a difference between the initial SoC for the at least two battery packs is greater than 5%, charging the at least two battery packs to 80% SoC.

14. The battery management method of claim 11, further comprising:
when a difference between the SoC for the at least two battery packs is greater than 3%, toggle charging the at least two battery packs to 80% SoC.

15. The battery management method of claim 11, further comprising:
During charging, when the SoCs for the at least two battery packs are greater than 80% and the difference between the SoCs for the at least two battery packs is less than 5%. stopping charging.
